(19) United States
(12) Patent Application Publication
(10) Pub. No.: US 2005/0214221 A1
(43) Pub. Date: Sep. 29, 2005

(54) Optical imaging probes

(75) Inventors: Kirtland G. Poss, Marblehead, MA (US); Karen N. Madden, Sudbury, MA (US); Ella Jones, Shrewsbury, MA (US); Sylvie Kossodo, Andover, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: VisEn Medical, Inc., Woburn, MA (US)

(21) Appl. No.: 10/938,744

(22) Filed: Sep. 10, 2004

Related U.S. Application Data
(63) Continuation of application No. PCT/US03/07579, filed on Mar. 11, 2003.
(60) Provisional application No. 60/363,499, filed on Mar. 11, 2002.

(51) Int. Cl. A61B 10/00

(52) U.S. Cl. 424/9.6

(57) ABSTRACT

This invention relates to optical imaging probes and the use of such probes for diagnosing and monitoring disease, and disease treatment. The optical imaging probes of the current invention can be used to identify and characterize normal and diseased tissues with regards to altered metabolic activity.

### RELATED APPLICATION

This application a continuation of International Application No. PCT/US03/07579, which designated the United States and was filed on Mar. 11, 2003, published in English, which claims the benefit of U.S. Provisional Application No. 60/363,499, filed on Mar. 11, 2002. The entire teachings of the above applications are incorporated herein by reference.

### BACKGROUND OF THE INVENTION

This invention relates to optical imaging probes and the use of such probes for diagnosing and monitoring disease, and for disease treatment. The optical imaging probes of the current invention can be used to identify and characterize normal and diseased tissues with regards to altered metabolic or physiologic activity.

With the sequencing of the human genome, there is an enormous effort underway to understand the precise molecular basis of different disease states. With this understanding of the molecular basis of different disease states comes the opportunity to non-invasively image specific molecular activity associated with normal and pathologic processes. The emerging field of molecular imaging has the ability to provide significantly more information about different disease states compared to traditional morphological or anatomical imaging alone. Traditional imaging techniques such as magnetic resonance (MR) imaging, computer tomography (CT), X-ray, and ultrasound (US) rely on physical parameters such as absorption, scattering, proton density, and relaxation rates as the primary source of contrast for disease detection. Specific molecular information using these modalities often cannot be obtained, or is of limited nature. Molecular imaging, however, uses specific molecular activity as the source of image contrast and therefore, can provide much more detailed information compared to traditional morphologic images. Such detailed understanding of disease states at their molecular level will help to (1) detect early disease, even before morphological changes are present, (2) better characterize different disease states, and (3) improve, guide, and monitor disease treatment.

Nuclear imaging using various radiolabeled molecules has demonstrated some clinical utility in being able to image certain forms of molecular activity. Various radiolabeled metabolite imaging probes are known in the art and the technique of using these radiolabeled metabolite imaging probes to image metabolic activity is well established. Specifically, this technique has been used successfully to label and image several different metabolites including deoxyglucose (Bar-Shalom et al., 2000, Semin. Nucl. Med. 30:150-185; and Yang et al., 2003, Radiology 226:465-473). PET imaging using [¹⁸F] fluorodeoxyglucose (FDG) is becoming a well-established clinical cancer imaging method that can be used to detect very small tumors and distant metastases, to help stage tumors, and to monitor a patient's response to therapy (Kubota, K., 2001, Ann. Nuc. Med. 15:471-486).

Although nuclear imaging of radioactively labeled metabolites has demonstrated some clinical utility, there remain significant limitations with these imaging approaches. Specifically, the short half-life of many radionuclides, including ¹⁸F, ¹¹C, ¹⁷O, and ^{99m}Tc, severely limits the time available for synthesis and subsequent imaging, and therefore any facilities using these technologies require skilled radiochemists on staff to synthesize the imaging agents immediately prior to use. In the case of PET imaging, a cyclotron is usually required on-site because of the extremely short half-life of most positron-emitting radionuclides, including ¹⁸F. In addition, the clinical hardware systems required to detect positron and gamma emitting radionuclides are relatively expensive and therefore, require a significant upfront capital investment. Because of these limitations, few clinical centers have the necessary expertise, resources, and money to operate a nuclear imaging center effectively.

Another significant disadvantage to nuclear imaging is that patients are exposed to radioactivity. Because strict clinical guidelines govern the amount of radiation a patient can receive over a given timeframe, the number of imaging procedures a patient can receive per year is limited. Therefore, nuclear imaging is limited for routine monitoring of a patient's disease state or response to therapy over time.

Molecular optical imaging is a new imaging modality that generates molecular images using penetrating light rays. Preferably, light in the red and near infrared range (600-1200 nm) is used to maximize tissue penetration and minimize absorption from natural biological absorbers such as hemoglobin and water. (See, e.g., Wyatt, 1997, Phil. Trans. R. Soc. London B 352:701-706; and Tromberg et al., 1997, Phil Trans. R. Soc. London B 352:661-667)

In near infrared fluorescence (NIRF) imaging, filtered light or a laser with a defined bandwidth is used as a source of excitation light. The excitation light travels through body tissues. When it encounters a NIRF molecule (“contrast agent”), the excitation light is absorbed. The NIRF then emits light that has detectably different properties (i.e., spectral properties of the probe (slightly longer wavelength), e.g., fluorescence) from the excitation light.

Various optical metabolite imaging probes have been developed for medical imaging. Most recently, near infrared fluorochromes (NIRFs) with preferential tissue distribution and greater hydrophilicity (Licha et al., 2000, Photochem. Photobiol. 72:392-398), receptor targeted fluorochromes (Becker et al., 2001, Nature Biotech. 19:327-331; and Bugaj et al. 2001, J Biomed. Opt. 6:122-133) and enzyme activatable optical probes have been described (Weissleder et al., 1999; Nature Biotech., 17:375-378; and Bremer et al., 2001, Nature Med., 7:743-748). Imaging using non-specific NIRFs such as those described by Licha et al. and indocyanine green, does not truly reflect differences in molecular or metabolic activity, as they primarily reflect differences in overall pharmacokinetics, vascular distribution (through differences in fluorochrome solubility and binding to plasma proteins) and excretion. While receptor targeted fluorochromes such as those described by Becker et al. and enzyme activatable probes such as those described by Weissleder et al. are able to image some forms of molecular activity, these probes are not optical metabolite imaging probes.

Several fluorescent derivatives of glucose have been described for in vitro use (Yamada et al. 2000, J Biol. Chem. 275:22278-22283; Molecular Probes, Eugene, Oregon; and U.S. Pat. No.5,877,310). Some of these reagents are used primarily to study glucose uptake into cells by microscopy. However, because these fluorescent agents do not absorb or emit light in the red or near infrared range, their in vivo use is very limited, i.e., for cancer detection in deep tissues. NIRFs are important to use compared to other fluorochromes because imaging of deeper tissues (>500 μm to 15 cm) requires the use of near infrared light. The other agents are used primarily as water soluble in vitro labeling reagents for proteins and nucleic acids for in vitro imaging applications such as flow cytometry.

Thus, there is a need in the art for in vivo optical metabolite imaging probes and imaging methods that are safer, less expensive, and more convenient than current nuclear imaging probes and methods. Furthermore, there is a need for non-radioactive metabolite imaging agents for applications in unique clinical situations where nuclear imaging is not a viable option including for reasons of resolution, during endoscopy, or in surgery and for repeatedly monitoring a patient's disease state over time.

### SUMMARY OF THE INVENTION

The invention is based on fluorochrome derivatized metabolically recognizable molecules that can be used as imaging agents for detection or evaluation of biological processes in vivo. Specifically, it has been found that near infrared fluorochromes (NIRFs) can be mono and polyvalently derivatized with metabolically recognizable molecules such that the resulting imaging probes can serve as imaging agents of metabolic and other biological processes in animal and humans. These optical metabolite imaging probes (termed “metabolite imaging probes” because they contain metabolically recognizable molecules) can be designed to have two unique features that enable imaging of metabolic and biological activities in vivo: 1) their preferable near infrared fluorescence enables effective tissue penetration for in vivo imaging, and 2) the “activity” (i.e., affinity for imaging metabolic processes) can be achieved by conjugating two or more metabolically recognizable molecules onto the fluorochrome structure (i.e., polyvalency). Thus, these optical metabolite imaging probes are ideal for in vivo imaging of metabolic alterations in mammals and humans.

The structure of the optical metabolite imaging probes (imaging agents) of the present invention can be described by the general formulas:
M(n)-F (I) or
M(n)-F-L(o) (II) or
M(n)-L(o)-F (III) or
L(o)-M(n)-F (IV)

Where:
M is a metabolically recognizable molecule;
Each of n and o is, independently, 1 to 30;

F is a fluorochrome molecule; and

L is another metabolically recognizable molecule or helper ligand to improve substrate binding and/or delivery.

The molecular weight of the optical imaging probe can be low (50-2,000 daltons) or high (above 2,000 daltons).

The metabolically recognizable molecules can be chemically linked to F, and can total 1-30 per entire optical imaging probe. In one embodiment, M is 2-30. In preferred embodiments, M is 2 or 3. The metabolically recognizable molecule itself may itself be polyvalent, i.e., have more than one repeating structural unit. After derivatization with a single reporter molecule, many metabolites remain metabolically active, but usually at lower rates compared to the underivatized metabolite. A key aspect of this present invention therefore relates to strategies to improve on metabolite or substrate activity in order to optimize imaging of metabolic alterations. While this can be achieved by: 1) optimizing linker systems, 2) rational design and ligand/target molecular modeling and 3) chemically modifying the substrate for optimized in vivo performance, degrees of polyvalency (including bivalency) can result in superior optical metabolite imaging probes with greater “activity” and affinity for imaging metabolic processes. Polyvalency is therefore often important to improve the “activity” and metabolic rates of derivatized NIRF imaging agents, and thus enhancing imaging of metabolic activity.

A “fluorochrome” includes, but is not limited to, a fluorochrome, a fluorophore, a fluorochrome quencher molecule, or any organic or inorganic dye. Preferred fluorochromes are red and near infrared fluorochromes (NIRFs) with absorption and emission maximum between 600 and 1200 nm. Preferred NIRFs have an extinction coefficient of at least 50,000 M^{-‘}cm^{31 1} in aqueous medium. Preferred NIRFs also have (1) high quantum yield (i.e., quantum yield greater than 5% in aqueous medium), (2) narrow excitation/emission spectrum, spectrally separated absorption and excitation spectra (i.e., excitation and emission maxima separated by at least 15 nm), (3) high chemical and photostability, (4) nontoxicity, (5) good biocompatibility, biodegradability and excretability, and (6) commercial viability and scalable production for large quantities (i.e., gram and kilogram quantities) required for in vivo and human use. Methods for measuring these parameters are known to one of skill in the art.

A “metabolically recognizable molecule” is any molecule produced, used, or recognized during metabolism. This includes, but is not limited to molecules produced, used, or recognized in carbohydrate metabolism, energy metabolism, fatty acid and lipid metabolism, nucleotide metabolism, amino acid metabolism, and co-factor and vitamin metabolism. (For current listing of metabolic pathways and metabolites please see Boehringer Mannheim Biochemical Chart at www.expasy.ch/cgi-bin/search-biochem-index.) (See also Salway, J., 1999, Metabolism at Glance, Blackwell Science Inc; 2nd ed.)

This includes, but is not limited to molecules such as carbohydrates (e.g., glucose, galactose, mannose, glycosaminoglycans, etc.), organic acids (e.g., lactate, citrate, tartrate, acetate, etc.), amino acids (e.g., methionine, tyrosine, glutamate, taurine, omithine, glutathione, etc.), halides (e.g., iodine, iodotyrosines chlorine, fluorine), steroids (e.g., estrogen, progesterone, testosterone, etc.), fatty acids (e.g., glycerol, palmitate, stearate, oleate, myrisates, etc.), lipids (e.g., cholesterol, phosphatidyl choline, ceramide, gangliosides, etc.), vitamins (e.g., thiamine, folate, biotin, riboflavin, niacin, etc.), nucleic acids and derivatives thereof (e.g., ATP, AMP, GTP, GMP, thiouracil, thymidine, urate, hypoxanthine, etc.), neurotransmitters (e.g., dopamine, serotonin, epinephrine, etc.), inorganic molecules (e.g., pyrophosphate, phosphate, phosphonates, sulfates, etc.), and drugs with proven action (e.g., therapeutic compounds).

A “metabolically recognizable molecule” also includes analogs of naturally occurring metabolically recognizable molecules. For instance, synthetic derivatives of natural metabolites such as phosphonate derivatives in which the P-O-P bond is replaced by a non-hydrolyzable or metabolizable P-C-P bond could be used in probes of this invention. This includes but is not limited to bisphosphonates such as etidronate, clodronate, pamidronate, alendronate, tiludronate, risedronate, ibandronate, zoledronate, incadronate, olpadronate, neridronate, oxidronate, and methylene diphosphonate (MDP).

Importantly, metabolically recognizable molecules such as small molecule drugs can also be used in this invention. For instance, many small molecule drugs are known in the art that are metabolically recognizable molecules, including drugs that are metabolically recognizable by the cytochrome P450 family of enzymes and by kinases, including serine, threonine, and tyrosine kinases. In one embodiment, the metabolically recognizable molecule is not, somatostatin, the somatostatin analog octreotate, or another somatostatin analog. In another embodiment, the metabolically recognizable molecule is not a matrix metalloprotease inhibitor.

Preferred metabolically recognizable molecules include, but are not limited to, deoxyglucose, thymidine, methionine, estradiol, danorubicin, acetate, dopamine, L-dopa, diprenorphine, methylspiperone, deprenyl, raclopride, phosphonates (e.g., methyldiphosphonates), tyrosine and methyltyrosines, glucoheptonate, folate, iodide, citrate, epinephrine, 1-amino-cyclobutane-1-carboxylic acid, arachidonic acid, palmitic acid, glycosyl-phosphatidylinositol, myristic acid, farnesyl diphosphate, triglycerides, misonidazole, choline, vitamin B6 and its derivatives, and topotecan.

In another embodiment, the optical metabolite imaging probe can become activated (i.e., have a change in detectable optical properties such as fluorescence intensity or wavelength shift) after being metabolized (i.e., a fluorescent pro-drug).

“Derivatized” means one or more metabolites chemically linked to the fluorochrome structure, where metabolically recognizable molecules may be chemically linked to the fluorochrome, and can total 1-30 per entire fluorochrome structure. Linkers or spacers may be used to chemically link the metabolically recognizable molecules, helper ligands or quenchers to the fluorochrome. Preferred embodiments are fluorochromes that are mono- or bivalently derivatized, but polyvalently (e.g., more than 3) derivatized fluorochromes are also featured in this invention. In addition, the metabolically recognizable molecule itself may itself be polyvalent, i.e., have more than one repeating structural unit. For example, a polysaccharide can be considered a repeating structural unit of a sugar molecule and a polypeptide can be considered a repeating structural unit of an amino acid. The monosaccharide units of a polysaccharide can be arranged in a linear or branched manner.

“Chemically linked” is meant connected by any attractive force between atoms strong enough to allow the combined aggregate to function as a unit. This includes, but is not limited to, chemical bonds such as covalent bonds (e.g., polar or non-polar), non-covalent bonds such as ionic bonds, metallic bonds, and bridge bonds, and hydrophobic interactions and van der Waals interactions.

A “helper ligand” is any moiety that can be chemically linked to the imaging probe of the present invention that enhances accumulation, targeting, binding, recognition, metabolic activity of the probe, or enhances the efficacy of the probe in any manner. This includes but is not limited to membrane (or transmembrane) translocation signal sequences, which could be derived from a number of sources including, without limitation, viruses and bacteria. Also included are moieties such as monoclonal antibodies (or antigen-binding antibody fragments, such as single chain antibodies) directed against a target-specific marker, a receptor-binding polypeptide directed to a target-specific receptor, a receptor-binding polysaccharide directed against a target-specific receptor and other molecules that target internalizing receptors including but not limited to nerve growth factor, oxytocin, bombesin, calcitonin, arginine vasopressin, angiotensin II, atrial natriuretic peptide, insulin, glucagons and glucagon-like peptides, prolactin, gonadotropin, and various opioids.

Derivatization of a fluorochrome may also change the biological properties of the NIRF itself. For instance, mono-, bi-, or polyvalent derivatization of a fluorochrome may improve the pharmacokinetics, toxicity, solubility, and fluorescence properties of the fluorochrome molecule itself, thereby making it a more suitable in vivo imaging agent, that could be used in any number of different applications which may or may not include imaging metabolic or physiologic activity.

The invention also features in vivo optical imaging methods. In one embodiment the method includes the steps of: (a) administering to a subject an optical imaging probe of the present invention; (b) allowing time for the optical imaging probe to reach the target tissue and, preferably, but not necessary, for molecules in the target tissue to metabolize the probe; (c) illuminating the target tissue with light of a wavelength absorbable by the optical imaging probe; and (d) detecting the optical signal emitted by the optical imaging probe.

These steps can also be repeated at predetermined intervals thereby allowing for the evaluation of emitted signal of the optical imaging probe in a subject or sample over time. The emitted signal may take the form of an image. The subject may be a mammal, including a human. The subject may also be non-mammalian, (i.e., C. elegans, drosophila, etc.). The sample can include, without limitation, cells, cell culture, tissue sections, cytospin samples, or the like. Similar methods can be carried out to perform in vitro imaging, for example on cell or tissue samples.

The invention also features an in vivo method for selectively detecting and imaging two or more optical metabolite imaging probes simultaneously. The method includes administering to a subject two or more optical metabolite imaging probes, either at the same time or sequentially, whose optical properties are distinguishable from that of the others. The method therefore allows the recording of multiple events or targets. Similar methods can be carried out to perform in vitro imaging, for example on cell or tissue samples.

The invention also features an in vivo method for selectively detecting and imaging one or more optical metabolite imaging probes, simultaneously with one or more targeted or activatable optical imaging probes, or magnetic resonance, CT, X-ray, ultrasound, or nuclear medicine imaging modalities or agents. The method includes administering to a subject one or more imaging probes, either at the same time or sequentially, including at least one optical metabolite imaging probe, whose properties are distinguishable from that of the others. The method therefore, allows the recording of multiple events or targets using more than one imaging modality or agent. Similar methods can be carried out to perform in vitro imaging, for example-on cell or tissue samples.

The methods of the invention can be used to determine a number of indicia, including tracking the localization of the optical imaging probe in the subject over time or assessing changes or alterations in the metabolism of the optical imaging probe in the subject over time. The methods can also be used to follow therapy for such diseases by imaging molecular events modulated by such therapy, including but not limited to determining efficacy, optimal timing, optimal dosing levels (including for individual patients or test subjects), and synergistic effects of combinations of therapy.

The invention can be used to help a physician or surgeon to identify and characterize areas of disease, such as colon polyps or vulnerable plaque, to distinguish diseased and normal tissue, such as detecting tumor margins that are difficult to detect using an ordinary operating microscope, e.g., in brain surgery, and help dictate a therapeutic or surgical intervention, e.g., by determining whether a lesion is cancerous and should be removed or non-cancerous and left alone.

The methods of the invention can also be used in the detection, characterization and/or determination of the localization of a disease, especially early disease, the severity of a disease or a disease-associated condition, the staging of a disease, and monitoring and guiding various therapeutic interventions, such as surgical procedures, and monitoring drug therapy. Examples of such disease or disease conditions include inflammation (e.g., inflammation caused by arthritis, for example, rheumatoid arthritis), all types of cancer (e.g., detection, assessing treatment efficacy, prognosis, characterization), cardiovascular disease (e.g., atherosclerosis and inflammatory conditions of blood vessels, ischemia, stroke, thrombosis), dermatologic disease (e.g., Kaposi's Sarcoma, psoriasis), ophthalmic disease (e.g., macular degeneration, diabetic retinopathy), infectious disease (e.g., bacterial, viral, fungal and parasitic infections), immunologic disease (e.g., Acquired Immunodeficiency Syndrome, lymphoma, multiple sclerosis, rheumatoid arthritis, diabetes mellitus), central nervous system disease (e.g., Parkinson's disease, Alzheimer's disease), and bone-related disease (e.g., osteoporosis, primary and metastatic bone tumors, osteoarthritis). Other diseases that can be assessed include neurodegenerative diseases, autoimmune diseases, inherited diseases, and environmental diseases. The methods of the invention can therefore be used, for example, to determine the presence of tumor cells and localization of tumor cells, the presence and localization of inflammation, the presence and localization of vascular disease including areas at risk for acute occlusion (vulnerable plaques) in coronary and peripheral arteries, regions of expanding aneurysms, unstable plaque in carotid arteries, and ischemic areas. The methods of the invention can also be used in identification of apoptosis, necrosis, and hypoxia.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of the Cy5.5-monovalent glucose probe.

FIG. 2 is a schematic diagram of the Cy5.5-bivalent glucose probe.

FIG. 3A is a scanned image of cellular uptake of monovalent glucose imaging probes in A431 tumor cells.

FIG. 3B is a scanned image of the inhibition of cellular uptake of monovalent glucose imaging probes by glucose in A431 tumor cells.

FIG. 3C is a scanned image of cellular uptake of bivalent glucose imaging probes in A431 tumor cells.

FIG. 3D is a scanned image of the inhibition of cellular uptake of bivalent glucose imaging probes by glucose in A431 tumor cells.

FIG. 4A is a scanned image of in vivo bivalent glucose imaging probes in tumor sites in an A431 tumor animal model.

FIG. 4B is a scanned image of in vivo monovalent glucose imaging probes in tumor sites in an A431 tumor animal model.

FIG. 4C is a scanned image of in vivo control (free Cy5.5) imaging probes in tumor sites in an A431 tumor animal model.

### DETAILED DESCRIPTION OF THE INVENTION

In one embodiment, the imaging agent (i.e., optical imaging probe) accumulates in diseased tissue at a different rate than in normal tissue. For example, the rate of accumulation of the agent can be at least 5%, 10%, 20%, 30%, 50%, 75%, or 90% faster in diseased tissue compared to normal tissue. Alternatively, the rate of accumulation of the agent can be at least 5%, 10%, 20%, 30%, 50%, 75%, or 90% slower in diseased tissue compared to normal tissue

In another embodiment, the imaging agent is metabolized in diseased tissue at a different rate than in normal tissue. For example, metabolism of the imaging agent can occur at a rate that is at least 5%, 10%, 20%, 30%, 50%, 75%, or 90% faster in diseased tissue compared to normal tissue. Alternatively, metabolism of the imaging agent can occur at a rate that is at least 5%, 10%, 20%, 30%, 50%, 75%, or 90% slower in diseased tissue compared to normal tissue.

In another embodiment, the imaging agent becomes trapped in cells.

In one embodiment the diseased tissue is cancerous and the imaging agent accumulates in malignant tissue at a different rate than in normal or benign tissue.

One preferred embodiment of the invention is based upon the well-accepted observation that malignant tissue may be easily distinguished from benign or normal tissue by its increased rate of glucose metabolism. Specifically, rapidly dividing cells have been shown to exhibit enhanced glucose metabolism, a requirement necessary to sustain their increased need for ATP generation and substrate storage. In addition to normal physiologically-related growth processes, cancer cell growth is heavily dependent upon increased glucose metabolism. Furthermore, the correlation between increased glucose metabolism and tumor growth has been well documented and exploited in the development of drugs aimed at blocking glucose metabolism for therapeutic purposes. Glucose transport across cell membranes requires the presence of specific integral membrane transport proteins, which includes the facilitative glucose carriers. Since the initial identification of the human erythrocyte glucose transporter, GLUT-1, more than 12 additional family members have been described and several have been shown to be overexpressed in various human cancers and cancer cell lines, leading to speculation that aberrant regulation of glucose metabolism and uptake by one or more transporter subtypes may correlate with tumor genesis.

For imaging of glucose metabolism, an optical metabolite imaging probe should be able to readily permeate the cell membrane and enter the cytosol. The optical metabolite imaging probe should also preferably be capable of interacting with specific enzymes involved in glucose metabolism. Many enzymes, receptors, and transporters are quite permissible. For example, GLUT-2, which normally helps transport glucose across the cell membrane, also recognizes and transports [¹⁹F]-deoxyglucose (FDG) and ^{99m}Tc-chelate-deoxyglucose. In addition, hexokinase, which is an enzyme that catalyzes the first step in glucose metabolism, (i.e., the phosphorylation of glucose to glucose-6-phosphate) is also quite permissible and can carry out this chemical reaction on FDG and ^{99m}Tc-chelate-deoxyglucose. Therefore, a preferred embodiment of the present invention for imaging glucose metabolism is comprised of 1-30 glucose or deoxyglucose molecules chemically linked to a suitable fluorochrome. Ideally, the imaging probe would become trapped in the cell. An optical metabolite glucose imaging probe could be used to diagnose and stage tumors, myocardial infarctions and neurological disease. In another embodiment, the metabolically recognizable molecule is not a sugar. In a preferred embodiment, 2 or 3 or more glucose or deoxyglucose molecules are chemically linked to a suitable fluorochrome.

Another preferred embodiment is based on the well-accepted observation that malignant tissue has a higher rate of cellular proliferation when compared to benign or normal tissue. The rate of cellular proliferation can be measured by determining the rate of DNA synthesis of cells, which can could be measured using nucleotide based metabolites such as thymidine. Thus, a preferred embodiment of the present invention for imaging cellular proliferation is comprised of 1-30 thymidine molecules, and analogs thereof, chemically linked to a suitable fluorochrome. In a preferred embodiment, 2 or 3 or more thymidine molecules are chemically linked to a suitable fluorochrome.

In another embodiment, the diseased tissue is in the central nervous system and the imaging agent is metabolized or accumulates in the diseased tissue at a different rate when compared to normal tissue. One preferred embodiment of the invention is based upon the well-accepted observation that the density of dopamine transporters and level of dopamine metabolism in the central nervous system is elevated or decreased in a number of different disease states including Parkinson's disease, Tourette's Syndrome, Lesch-Nyhan Syndrome, Rhett's Syndrome, and in substance abusers. Proper dopamine metabolism also is required to maintain a state of psychological well-being.

For imaging of increased or decreased levels of dopamine transporters and level of dopamine metabolism, an optical metabolite imaging probe should be able to readily bind to the dopamine transporter (DAT) and, ideally, enter the cytosol of the cell. The dopamine transporter is known to bind to and transport a wide range of metabolites including L-dopa and tropanes. Therefore, these metabolites could be used to image increased or decreased levels of dopamine transporters and dopamine metabolism. Thus, a preferred embodiment of the present invention for imaging increased or decreased levels of dopamine transporters and level of dopamine metabolism, is comprised of 1-30 L-dopa, dopamine, tropane or raclopride molecules, or combinations thereof, chemically linked to a suitable fluorochrome. In addition, preferred brain imaging agents of the present invention also have blood brain barrier permeability. In a preferred embodiment, 2 or 3 or more L-dopa, dopamine, tropane or raclopride molecules, or combinations thereof are chemically linked to a suitable fluorochrome.

In another embodiment, the diseased tissue is in the cardiovascular system and the imaging agent is metabolized or accumulates in the diseased tissue at a different rate when compared to normal tissue. One preferred embodiment of the invention is based upon the well-accepted observation that many common cardiac disorders are the result of imbalances of myocardial metabolism. Oxidation of long chain fatty-acids is the major energy pathway in myocardial tissue and abnormal rates of cellular uptake, synthesis and breakdown of long-chain fatty acids are indicative of various cardiac diseases including coronary artery disease, myocardial infarction, cardiomyopathies, and ischemia (Railton et al., 1987 Euro. J NucL. Med. 13:63-67; and Van Eenige et al., 1990 Eur. HeartJ. 11:258-268).

For imaging of increased or decreased levels of cellular uptake, synthesis and breakdown of long-chain fatty acids in vascular disease, an optical metabolite imaging probe should be able to permeate the cell membrane and enter the cytosol and, preferably, interact with enzymes involved in long-chain fatty acid metabolism. Fatty acids generally enter cells via passive diffusion. After cellular entry, many fatty acids undergo β-oxidation, which is catalyzed by coenzyme A synthetase. Therefore, a preferred embodiment of the present invention for imaging cardiovascular disease is comprised of 1-30 fatty acid molecules chemically linked to a suitable fluorochrome. In a preferred embodiment, 2 or 3 or more fatty acid molecules are chemically linked to a suitable fluorochrome.

Another preferred embodiment of the invention is based upon the well-accepted observation that imbalances in osteoblast activity is indicative of several disease states including osteoporosis, osteoblastic cancer metastases, early calcification in atherosclerosis and cancer lesions, arthritis and otoslcerosis. Phosphonates and analogs thereof localize in areas where osteoblast activity is high, including areas of active bone remodeling (Zaheer et al., 2001, Nature Biotech 19:1148-1154). Thus, a preferred embodiment of the present invention for imaging bone diseases and also atherosclerosis and otoslcerosis is comprised of 1-30 methylene diphosphonate, pyrophosphate, and/or alendronate molecules chemically linked to a suitable NIRF. In a preferred embodiment, 2 or 3 or more methylene diphosphonate, pyrophosphate, and/or alendronate molecules are chemically linked to a suitable fluorochrome.

Another preferred embodiment of the invention is based upon the well-accepted observation that tumors and infarcted regions are hypoxic when compared to normal or unaffected tissue. Compounds such as nitroimidizoles, such as misonidazole, are known in the art that preferentially accumulate and are retained in hypoxic areas. In cells with reduced oxygen content, these compounds are metabolized by cellular reductases, such as xanthine oxidase, and subsequently become trapped inside the cell. Therefore, a preferred embodiment of the present invention for imaging hypoxia is comprised of 1-30 misonidazole molecules chemically linked to a suitable fluorochrome structure. In a preferred embodiment, 2 or 3 or more misonidazole molecules are chemically linked to a suitable fluorochrome.

In another embodiment the optical imaging probe could also be represented by the following general formulas (V) and (VI):
F-M-F (V) or
F-M-Q (VI)

where:

M is a metabolically recognizable molecule;

F is a fluorchrome molecule; and

Q is a quencher molecule.

In this embodiment, the optical imaging probe could be activatable, where the probe in its native state has little or no fluorescence emission and detection of the probe is not possible until it has been activated or metabolized. In a preferred embodiment M is a peptide or nucleic acid sequence.

A “quencher” molecule is any molecule that when appropriately interacting with the fluorochrome molecule quenches the optical properties of the fluorochrome molecule. This includes but is not limited to quenchers available and known to those skilled in the art such as DABCYL, QSY-7, QSY-33 (Molecular Probes, Eugene, Oreg.), fluorescein isothiocyanates (FITC) and rhodamine pair (Molecular Probes, Eugene, Oreg.).

In the practice of the present invention, the metabolically recognizable molecule, helper ligand, or quencher can be chemically linked to the fluorochrome by any method presently known in the art for chemically linking two or more moieties; this includes but is not limited to the use of linker or spacer moieties. Useful linker moieties include both natural and non-natural amino acids and nucleic acids, as well as synthetic linker molecules. In preferred embodiments of the present invention, isothiocyanate, isocyanate, and hydroxysuccinimide ester or hydroxysulfosuccinimide ester functionalities on the fluorochrome are reacted with amino functional groups on the metabolically recognizable molecule, helper ligand, or linker or spacer moiety to form a suitable chemical linkage.

Various fluorochromes are described in the art and can be used to construct optical metabolite imaging probes according to this invention. These fluorochromes include but are not limited to cyanine, hemi-cyanine, azacarbocyanine, sulfo-benze-indocyanine, squarain, benzopyrylium-polymethine, and 2- or 4- chromenyliden based merocyanine dyes.

Exemplary fluorochromes include the following: Cy5.5, Cy5, and Cy7 (Amersham Biosciences, Piscataway, N.J.); IRD38 and IRD78 (LI-COR, Lincoln, Nerb.); NIR-1 and IC5-OSu, (Dojindo, Kumamoto, Japan); AlexaFluor 660 and AlexaFluor 680, (Molecular Probes, Eugene, Oreg.); FAR-Blue, FAR-Green One, and FAR-Green Two (Innosense, Giacosa, Italy), ADS 790-NS and ADS 821-NS (American Dye Source, Montreal, Canada), Atto680 (Atto-Tec, Siegen, Germany), DY-680, DY-700, DY-730, DY-750, DY-782, (Dyomics, Jena, Germany), EVOBlue (Evotec, Hamburg, Germany) and indocyanine green (ICG) and its analogs and derivatives (Licha et al., 1996, SPIE 2927:192-198; US 5,968,479), and indotricarbocyanine (ITC; WO 98/47538). Other examples of exemplary fluorochromes include Cy7.5 (Amersham Biosciences, Piscataway, N.J.), AlexaFluor 700 and AlexaFluor 750 (Molecular Probes, Eugene, Oreg.), FAR 5.5 (Innosense, Giacosa, Italy), fluorescent quantum dots (zinc sulfide-capped cadmium selenide nanocrystals) (QuantumDot Corporation, Hayward, Calif.), NIR2, NIR3, and NIR4 (Lin et al., 2002 Bioconj. Chem. 13:605-610) and chelated lanthanide compounds. Fluorescent lanthanide metals include europium and terbium. Fluorescence properties of lanthanides are described in Lackowicz, 1999, 15 Principles of Fluorescence Spectroscopy, 2nd Ed., Kluwar Academic, New York.

Fluorochromes that can be used to construct optical metabolite imaging probes are also described in U.S. Pat. Application No. 2002/0064794, PCT Publication No. WO 02/24815, U.S. Pat. No. 5,800,995, U.S. Pat. No. 6,027,709, PCT Publication No. WO 00/53678, PCT Publication No. WO 01/90253, EP 1273584, U.S. Patent Application No. 2002/0115862, EP 1065250, EP1211294, EP 1223197, PCT Publication No. WO 97/13810, U.S. Pat. No. 6,136,612, U.S. Pat. No. 5,268,486, U.S. Pat. No. 5,569,587, and Lin et al., 2002 Bioconj. Chem. 13:605-610, the entire teachings of which are incorporated herein by reference.

Table 1 summarizes information on the properties of several exemplary fluorochromes that can be used in the present invention.

TABLE 1
Exemplary Fluorochromes

| | | λex | λem |
|---|---|---|---|
| Fluorochrome | Source | (nm) | (nm) |
| Cy5.5 | Amersham | 675 | 694 |
| Cy7 | Amersham | 747 | 776 |
| Far-Blue | Innosense | 660 | 678 |
| Far- Green One | Innosense | 800 | 820 |
| Far- Green Two | Innosense | 772 | 788 |
| IRDye38 | Li-COR | 778 | 806 |
| IRDye78 | Li-COR | 768 | 796 |
| AlexaFluor 680 | Molecular Probes | 679 | 702 |
| AlexaFluor 700 | Molecular Probes | 702 | 723 |
| AlexaFluor 750 | Molecular Probes | 749 | 775 |
| DY-680 | Dyomics | 662 | 699 |
| DY-700 | Dyomics | 702 | 723 |
| DY-730 | Dyomics | 722 | 748 |

In preferred embodiments of the present invention, the in vivo half-life of the optical imaging probe is at least 10 minutes, but more preferable at least 30 minutes to 1 hour. In other preferred embodiments of the invention, the in vivo half-life of the optical imaging probe is greater than one hour. Methods for assessing the half-life of probes are known to those skilled in the art. In other preferred embodiments of the present invention, the optical imaging probes show little serum protein binding affinity.

In another embodiment of the present invention, the optical imaging probes can be manufactured into an acceptable pharmaceutical formulation.

Pharmaceutically acceptable carriers, adjuvants, and vehicles may be used in the composition or pharmaceutical formulation of this invention. Included carriers, adjuvants, or and vehicles include, but are not limited to, ion exchangers, alumina, aluminum stearate, lecithin, serum proteins such as albumin, buffer substances such as phosphate, glycine, sorbic acid, potassium sorbate, TRIS (tris(hydroxymethyl)amino methane), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block polymers, sugars such as glucose, and suitable cryoprotectants.

The pharmaceutical compositions of the invention may be in the form of a sterile injectable preparation. This preparation can be prepared by those skilled in the art of such preparations according to techniques known in the art. The possible vehicles or solvents that can be used to make injectable preparations include water, Ringer's solution, and isotonic sodium chloride solution, and D5W. In addition, oils such as mono- or di-glycerides and fatty acids such as oleic acid and its derivatives can be used. The pharmaceutical compositions of the present invention may also be in the form of a salt.

The formulation of the probe can also include an antioxidant or some other chemical compound that prevents or reduces the degradation of the baseline fluorescence, or preserves the fluorescence properties, including, but not limited to, quantum yield, fluorescence lifetime, and excitation and emission wavelengths. These antioxidants or other chemical compounds can include, but are not limited to, melatonin, dithiothreitol (dTT), defroxamine (DFX), methionine, DMSO, and N-acetyl cysteine.

The probes and pharmaceutical compositions of the present invention can be administered orally, parentally, by inhalation, topically, rectally, nasally, buccally, vaginally, or via an implanted reservoir. The term “parental administration” includes intravenous, intramuscular, subcutaneous, intraarterial, intraarticular, intra synovial, intrastemal, intrathecal, intraperitoneal, intracisternal, intrahepatic, intralesional, intracranial and intralymphatic injection or infusion techniques. The probes may also be administered via catheters or through a needle to any tissue.

For ophthalmic use, the pharmaceutical composition of the invention may be formulated as micronized suspensions in isotonic, pH adjusted sterile saline. Alternatively, the compositions can be formulated in ointments such as petrolatum.

For topical application, the new pharmaceutical compositions can also be formulated in a suitable ointment, such as petrolatum. Transdermal patches can also be used. Topical application for the lower intestinal tract or vagina can be achieved by a suppository formulation or enema formulation.

In preferred embodiments of the present invention, the optical imaging probe is water soluble (i.e., has a n-octanol-water distribution coefficient being less than 2.0 and is non-toxic (i.e., has an LD₅₀ of greater than 50mg/kg body weight or higher). In other preferred embodiments of the present invention, the optical imaging probes do no have any phototoxic properties.

Although the invention involves novel optical imaging probes, general principles of fluorescence, optical image acquisition, and image processing can be applied in the practice of the invention. For a review of optical imaging techniques, see, e.g., Alfano et al., 1997, Ann. NY Acad. Sci., 820:248-270.

An imaging system useful in the practice of this invention typically includes three basic components: (1) an appropriate light source for fluorochrome excitation, (2) a means for separating or distinguishing emissions from light used for fluorochrome excitation, and (3) a detection system. This system could be hand-held or incorporated into other useful imaging devices such as surgical goggles or intraoperative microscopes.

Preferably, the light source provides monochromatic (or substantially monochromatic) near infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., 1994, Proc. Natl. Acad Sci. USA 91:4887-4891; Ntziachristos et al;, 2000, Proc. Natl. Acad. Sci. USA 97:2767-2772; and Alexander, 1991, J Clin. Laser Med. Surg. 9:416-418. Information on near infrared lasers for imaging can be found at http://www.imds.com and various other well-known sources.

A high pass or bandpass filter can be used to separate optical emissions from excitation light. A suitable high pass or bandpass filter is commercially available from Omega Optical.

In general, the light detection system can be viewed as including a light gathering/image forming component and a light detection/image recording component. Although the light detection system may be a single integrated device that incorporates both components, the light gathering/image forming component and light detection/image recording component will be discussed separately.

A particularly useful light gathering/image forming component is an endoscope. Endoscopic devices and techniques that have been used for in vivo optical imaging of numerous tissues and organs, including peritoneum (Gahlen et al., 1999, J. Photochem. Photobiol.B 52:131-135), ovarian cancer (Major et al., 1997, Gynecol. Oncol. 66:122-132), colon and rectum (Mycek et al., 1998, Gastrointest. Endosc. 48:390-394; Stepp et al., 1998, Endoscopy 30:379-386), bile ducts (Izuishi et al., 1999, Hepatogastroenterology 46:804-807), stomach (Abe et al., 2000, Endoscopy 32:281-286), bladder Kriegmair et al., 1999, Urol. Int. 63:27-31; Riedl et al., 1999, J. Endourol. 13:755-759), lung (Hirsch et al., 2001, Clin. Cancer Res. 7:5-220), and brain (Ward,1998, J. Laser Appl. 10:224-228) can be employed in the practice of the present invention.

Other types of light gathering components useful in the invention are catheter-based devices, including fiber optics devices. Such devices are particularly suitable for intravascular imaging. See, e.g., Teamey et al., 1997, Science 276:2037-2039; and Teamey et al. 1996 Circulation 94:3013.

Still other imaging technologies, including phased array technology (Boas et al., 1994, Proc. Natl. Acad. Sci. USA 91:4887-4891; Chance, 1998, Ann. NYAcad. Sci. 838:29-45), optical tomography (Cheng et al., 1998, Optics Express 3:118-123; Siegel et al., 1999, Optics Express 4:287-298), intravital microscopy (Dellian et al., 2000, Br. J. Cancer 82:1513-1518; Monsky et al, 1999, Cancer Res. 59:4129-4135; Fukumura et al., 1998, Cell 94:715-725), confocal imaging (Korlach et al., 1999, Proc. Natl. Acad. Sci. USA 96:8461-8466; Rajadhyaksha et al., 1995, J Invest. Dermatol. 104:946-952; Gonzalez et al., 1999, J Med. 30:337-356), and fluorescence mediated tomography (Nziachristos et al., 2002, Nature Medicine 8:757-760) can be employed in the practice of the present invention.

Any suitable light detection/image recording component, e.g., charge coupled device (CCD) systems or photographic film, can be used in the invention. The choice of light detection/image recording will depend on factors including type of light gathering/image forming component being used. Selecting suitable components, assembling them into a near infrared imaging system, and operating the system is within ordinary skill in the art.

Importantly, the compositions and methods of the present invention may be used in combination with other imaging compositions and methods. For example, the methods of the present invention may be used in combination with other traditional imaging modalities such as X-ray, CT, PET, SPECT, and MRI. For instance, the compositions and methods of the present invention may be used in combination with CT and MRI to obtain both anatomical and metabolic information simultaneously. The compositions and methods of the present invention may also be used in combination with X-ray, CT, PET, SPECT, and MR contrast agents or the optical imaging probes of the present inventions may also contain components, such as iodine, gadolidium atoms, and radioactive isotopes, which can be detected using CT, PET, SPECT, and MR imaging modalities in combination with optical imaging. The optical imaging probes of the present invention may be also be constructed using molecules with various magnetic properties, such as iron oxide nanoparticles. These dual optical/MR imaging probes can be used for imaging not only the metabolic activity of a variety of different disease states by measuring the optical signal, but also their precise localization from their effects on T2 weighted MR images (Josephson et al., 2002 Bioconj. Chem. 13:554-560).

### EXEMPLIFICATION

Synthesis of a Cy5.5 Monovalent Glucose Optical Imaging Probe

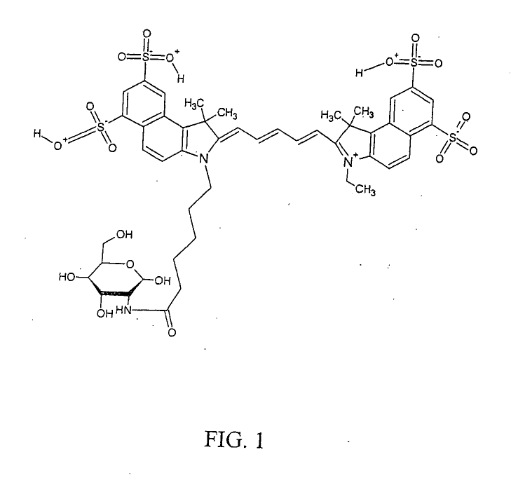

Synthesis of a monovalent NIRF-glucose probe was initially performed with glucosamine and a commercially-available fluorochrome, Cy5.5 (FIG. 1). Glucosamine (32 mg, 148 μmole dissolved in DMSO) was added to triethylamine (15 mg, 148 μmole) and the reaction continued for 10 minutes. Cy5.5-mono-N-hydroxysuccinimide ester (Cy5.5-mono-NHS ester) (1 mg, 886 nmole; Amersham) was dissolved in a minimum amount of dimethyl sulfoxide (DMSO) and added drop-wise to the glucosamine solution. The reaction mixture was stirred for 24 hours, and the resulting product purified by “dry flash” column chromatography with acetonitrile as the mobile phase. The product was extracted with diethyl ether, re-dissolved in water and lyophilized. A purified product with molecular formula of C₄₇ H₅₅ N₃ O₁₈ S₄ and corresponding [M+H]⁺ mass unit of 1078 was obtained by ESI-MS (electrospray ionization mass spectrometry). The overall yield of this probe based on Cy5.5 absorbance was determined to be 497 umoles.

Synthesis of a Cy5.5 Bivalent Glucose Optical Imaging Probe

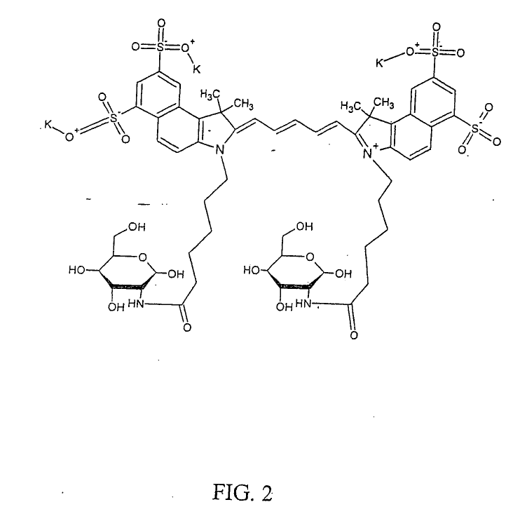

Synthesis of a monovalent NIRF-glucose probe was performed with glucosamine and a commercially-available fluorochrome, Cy5.5 (FIG. 2). Briefly, glucosamine (200 mg, 900 μmole dissolved in DMSO) was added to triethylamine (100 mg, 1000 μmole), and the reaction allowed to continue for 10 minutes. Commercially-available Cy5.5-bis-NHS ester was dissolved in minimum amount of DMSO and added drop wise. The resulting reaction mixture was stirred for 24 hours and the reaction product purified by “dry flash” column chromatography with acetonitrile.

Cy5.5 Glucose Optical Imaging Probe Uptake in Cell Culture

The human epidermoid carcinoma A431 cell line is known to express high levels of the facilitative glucose transporter GLUT-1 and has been shown to produce subcutaneous tumors with high efficiency in immunologically compromised mice. The A431 cell line was obtained from the American Type Culture Collection and grown in DMEM with 4.5 g/l glucose, supplemented with 10% fetal bovine serum (Life Technologies, NY) and cultured in a humidified atmosphere containing 5% CO₂ and 95% air at 37° C.

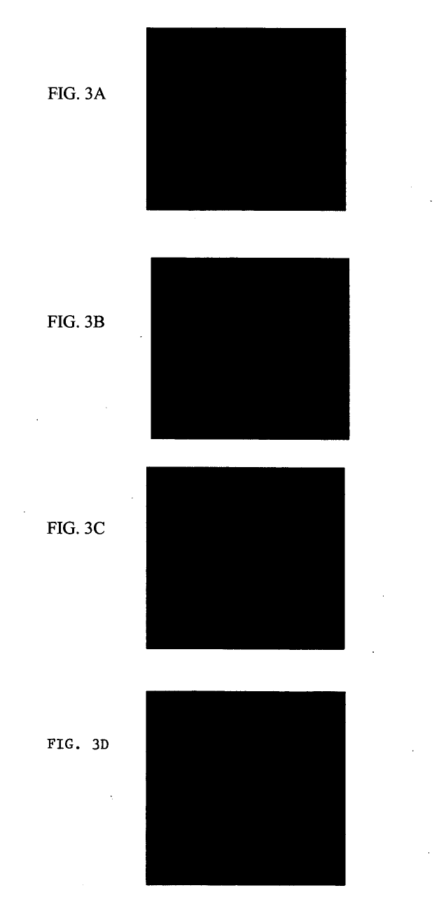

Utilizing the monovalent and bivalent Cy5.5-conjugated glucose probes, in vitro uptake experiments were performed by incubating A431 cells with 0.1 mM or 1 mM of each glucose probe for 30 minutes in glucose-free DMEM. After removal of the medium, cells were rinsed with ice-cold phosphate buffered saline (PBS) in preparation for microscopy. Excitation and emission filters (647/680) were utilized for the detection of Cy5.5. FIGS. 3A and 3C demonstrates that at 1 mM concentration, the Cy5.5-conjugated glucose probes are taken up by A431 cells, confirming in vitro uptake of the glucose probes as shown by fluorescence confocal microscopy.

Utilizing the monovalent and bivalent Cy5.5-conjugated glucose probes, in vitro uptake competition experiments were performed by incubating A431 cells with 1 mM (monovalent) or 0.7 mM (bivalent) of each probe, for 30 minutes, in the presence of 50 and 100 mM glucose, respectively. After removal of the medium, cells were rinsed with ice-cold PBS and visualized under confocal microscopy. FIGS. 3B and 3D demonstrate that glucose inhibits cellular uptake of the Cy5.5-conjugated monovalent (1mM) or bivalent (0.7 mM) probes, thus demonstrating that the cellular uptake of the probe occurs via glucose transporters. Under the same conditions, free Cy5.5 uptake by A431 cells was not inhibited by incubation with glucose.

In Vivo Cv5.5 Glucose Optical Imaging Probe Cell Uptake

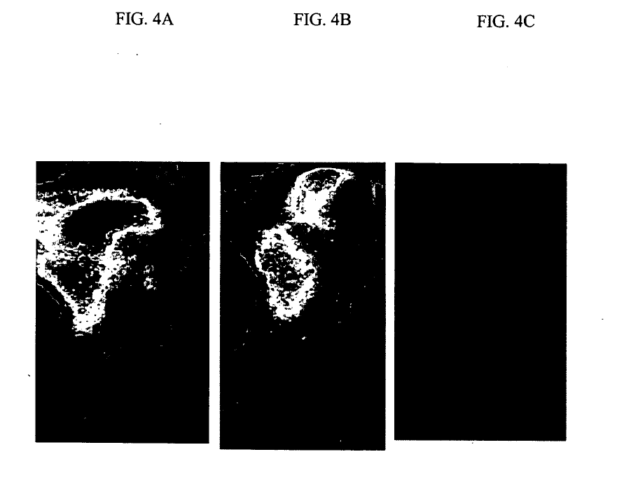

A431 carcinoma cells grown in culture were trypsinized, washed and resuspended in PBS at a density of 2×10⁷ cells/ml. Female Balb-c nu/nu athymic mice (6-8 weeks of age) received bilateral subcutaneous injections with 2×106⁶ cells (100 μl cell suspension) in the mammary fat pads of the first or second mammary glands. Tumors were allowed to grow until a target diameter of 3 mm ×3 mm (volume =13.5 mm³) was obtained. After requisite tumor sizes were reached, animals received an intravenous tail vein injection with 10 nmoles (based upon fluorochrome absorbance) of either the monovalent and bivalent glucose probes. Mice were anesthetized prior to imaging and imaged at 2, 15, 45, and 60 minutes. Imaging was performed using a custom built reflectance imaging system. In this imaging system set-up, a 150 W halogen light source was used to provide broad spectrum white light. A removable band pass optical filter (630RDF30, Omega Optical) was mounted between the bulb and a fiber optic bundle to create a uniform excitation source in the 610 to 650 rn range. Two mirrors were used to direct the light path to the imaging object and/or to the detector. Photons emanating from the fluorescent imaging object were selected using a 700 nm long pass filter. The filter was effective in removing scattered excitation photons, partially due to the wide frequency separation of the filter set. The bandpass excitation filter was mounted in a removable holder and the emission filter was mounted on a flywheel to allow for easy switching between fluorescent imaging and white light imaging, without moving the animal. The NIRF signal was detected by a low light level CCD and the signal output was recorded on a PC computer as 12 bit data using Kodak 1D imaging software. The imaging results are shown in FIGS. 4A (bivalent probe), 4B (monovalent probe) and 4C (free Cy5.5) and demonstrate that the glucose probes accumulate and enhance the tumor sites within minutes of probe injection as compared to the control probe (free Cy5.5).

Synthesis of Cv7, Alexa Fluor 750 , and NIR2 Monovalent Glucose Optical Imaging Probes

Glucosamine (32 mg, 148 μmole dissolved in DMSO) is added to triethylamine (15 mg, 148 μmole), and the reaction is allowed to continue for 10 minutes. Monoflnctional NHS ester fluorochrome derivatives of Cy7, Alexa Fluor 750, or NIR2 (approximately 1 mg, 900 nmole) are dissolved in a minimal amount of DMSO and added drop-wise to the glucosamine solution. The resulting reaction mixture is stirred for 24 hours, and the product purified by either “dry flash” column chromatography with acetonitrile as the mobile phase or reverse phase HPLC. The product will be extracted with diethyl ether, re-dissolved in water and lyophilized.

Synthesis of Cy7 Bivalent Glucose Optical Imaging Probe

Glucosamine (200 mg, 900 μmole dissolved in DMSO) is allowed to react with triethylamine (100 mg, 1000 μmole) for 10 minutes. Commercially-available Cy7-bis-NHS ester (approximately 5 mg, 4 μmole, Amersham) is dissolved in a minimum amount of DMSO and added drop-wise to the glucosamine solution. The resulting reaction mixture is stirred for 24 hours and the reaction product purified by either “dry flash” column chromatography with acetonitrile or reverse phase HPLC.

Synthesis of a Cy5.5 and Cy7 Bivalent Folate Optical Imaging Probe

Folatic acid is converted to an activated ester by reacting with N-hydroxysuccinimide in DMF using dicyclohexylcarbodiimide (DCC) as the condensing agent. 2,2′-(ethylenedioxy)bis-ethylamine (EDBEA) is then attached to the activated folate ester; thus forming an amino functional group on the folate molecule to which commercially-available Cy5.5-bis-NHS ester and Cy7- bis-NHS ester is then reacted. Briefly, 477 mg (1 mmole) of folic acid dihydrate, 15 ml of anhydrous DMSO, 0.31 ml (2 mmole) of DCC and 230 mg (2 mmole) of NHS is combined in a flask and heated at 50° C. for several hours. After cooling the mixture to room temperature, 1 ml of diisopropylamine and 1.5 ml of EDBEA are added and mixture stirred at room temperature for 24 hours. Acetonitrile is then added to precipitate the desired product. The product is washed with ethyl acetate, dried under vacuum, and then purified by either “dry flash” column chromatography or reverse phase HPLC.

The resulting amino functionalized folate is then reacted with commercially-available Cy5.5-bis-NHS ester or Cy7-bis-NHS ester. Approximately 5mg of either Cy5.5-bis-NHS ester or Cy7-bis-NHS ester are dissolved in a minimal amount of DMSO and added drop-wise to a solution containing the amino functionalized folate molecule (4mg of the amino functionalized folate molecule dissolved in 0.3 ml of 0.1 M NaHCO₃). The resulting reaction mixture is stirred for 24 hours and the reaction product purified by either “dry flash” column chromatography with acetonitrile or reverse phase HPLC.

1. An optical imaging probe represented by general formula (I):
M(ₙ)-F (I)
wherein:
M is a metabolically recognizable molecule;
n is 2to 30; and
F is a fluorochrome molecule.

2. The probe of claim 1, wherein M is selected from the group consisting of glucose, deoxyglucose, L-dopa, dopamine, thymidine, methionine, estradiol, acetate, raclopride, methyldiphosphonate, folate, a long-chain fatty acid, misonidazole, and a therapeutic compound.

3. The probe of claim 1, wherein n is 2.

4. The probe of claim 1, wherein n is 3.

5. The probe of claim 1, wherein the fluorochrome molecule has absorption and emission maximum between 600 nm and 1200 nm

6. The probe of claim 1, wherein the fluorochrome molecule is selected from the group consisting of Cy5.5, Cy7, Alexa Fluor 680, and NIR1.

7. The probe of claim 1, wherein the fluorochrome molecule is selected from the group consisting of Cy7.5, Alexa Fluor 700, Alexa Fluor 750, and NIR2.

8. An optical imaging probe represented by general formula II, III, or IV:
M(ₙ)-F-L(ₒ), (II), or
M(ₙ)-L(ₒ)-F (III), or
L(ₒ)-M(ₙ)-F (IV)
wherein:
M is a metabolically recognizable molecule;
each n or o is, independently, 1 to 30;
F is a fluorochrome molecule; and
L is another M or a helper ligand.

9. The probe of claim 8, wherein M is selected from the group comprising of glucose, deoxyglucose, L-dopa, dopamine, thymidine, methionine, estradiol, acetate, raclopride, methyldiphosphonate, folate, a long-chain fatty acids, misonidazole, and a therapeutic compound.

10. The probe of claim 8, wherein n is 1.

11. The probe of claim 8, wherein n is 2.

12. The probe of claim 8, wherein n is 3.

13. The probe of claim 8, wherein o is 1.

14. The probe of claim 8, wherein o is 2.

15. The probe of claim 8, wherein o is 3.

16. The probe of claim 8, wherein the fluorochrome molecule has absorption and emission maximum between 600 nm and 1200 nm

17. The probe of claim 8, wherein the fluorochrome molecule is selected from the group consisting of Cy5.5, Cy7, Alexa Fluor 680, and NIR1.

18. The probe of claim 8, wherein the fluorochrome molecule is selected from the group consisting of Cy7.5, Alexa Fluor 700, Alexa Fluor 750, and NIR2.

19. The probe of claim 8, wherein the helper ligand is selected from the group comprising a membrane translocation signal sequence, an antibody, an antibody fragment, a receptor-binding polypeptide, a polypeptide, and a receptor-binding polysaccharide.

20. A method of in vivo optical imaging, the method comprising:
(a) administering to a subject an optical imaging probe of claim 1;
(b) allowing time for the optical imaging probe to reach the target tissue;
(c) illuminating the target tissue with light of a wavelength absorbable by the optical imaging probe; and
(d) detecting the optical signal emitted by the optical imaging probe.

21. The method of claim 20, wherein steps (a)-(d) are repeated at predetermined intervals thereby allowing for evaluation of emitted signal of the optical imaging probe in the subject over time.

22. The method of claim 20, wherein the signal emitted by the optical imaging probe is used to construct an image.

23. The method of claim 22, wherein the image is co-registered with an image obtained by magnetic resonance or computed tomography imaging.

24. The method of claim 20, wherein the subject is an animal.

25. The method of claim 20, wherein the subject is a human.

26. The method of claim 20, wherein the illuminating and detecting steps are done using an endoscope, catheter, tomographic systems, hand-held optical imaging systems, surgical goggles, or intraoperative microscope.

27. The method of claim 20, wherein the presence, absence, or level of optical signal emitted by the optical imaging probe is indicative of a disease state.

28. The method of claim 20, wherein the method is used in the early detection or staging of a disease.

29. The method of claim 20, wherein the method is used in monitoring or dictating a therapeutic course of action for a treatment of a disease.

30. The method of claim 20, wherein the method is used to assess the effect of one or more therapies on a disease state.

31. The method of claim 30, wherein the disease is selected from the group consisting of cancer, a cardiovascular disease, a neurodegenerative disease, an immunologic disease, an autoimmune disease, an inherited disease, an infectious disease, a bone disease, and an environmental disease.

32. The method of claim 20, wherein in step (a), more than one distinguishable optical imaging probe is administered to the subject and wherein in step (d) more than one optical signal emitted by the optical imaging probes target is detected.

33. A method of in vivo optical imaging, the method comprising:
(a) administering to a subject an optical imaging probe of claim 7;
(b) allowing time for the optical imaging probe to reach the target tissue:
(c) illuminating the target tissue with light of a wavelength absorbable by the optical imaging probe; and
(d) detecting the optical signal emitted by the optical imaging probe.

34. The method of claim 33, wherein steps (a)-(d) are repeated at predetermined intervals thereby allowing for evaluation of emitted signal of the optical imaging probe in the subject over time.

35. The method of claim 33, wherein the signal emitted by the optical imaging probe is used to construct an image.

36. The method of claim 35, wherein the image is co-registered with an image obtained by magnetic resonance or computed tomography imaging.

37. The method of claim 33, wherein the subject is an animal.

38. The method of claim 33, wherein the subject is a human.

39. The method of claim 33, wherein the illuminating and detecting steps are done using an endoscope, catheter, tomographic systems, hand-held optical imaging systems, surgical goggles, or intraoperative microscope.

40. The method of claim 33, wherein the presence, absence, or level of optical signal emitted by the optical imaging probe is indicative of a disease state.

41. The method of claim 33, wherein the method is used in the early detection or staging of a disease.

42. The method of claim 33, wherein the method is used in monitoring or dictating a therapeutic course of action for a treatment of a disease.

43. The method of claim 33, wherein the method is used to assess the effect of one or more therapies on a disease state.

44. The method of claim 43, wherein the disease is selected from the group consisting of cancer, a cardiovascular disease, a neurodegenerative disease, an immunologic disease, an autoimmune disease, an inherited disease, an infectious disease, a bone disease, and an environmental disease.

45. The method of claim 33, wherein in step (a), more than one distinguishable optical imaging probe is administered to the subject and wherein in step (d) more than one optical signal emitted by the optical imaging probes target is detected.

46. An optical imaging probe represented by general formula (I):
M(ₙ)-F (I)
wherein:
M is a metabolically recognizable molecule selected from the group consisting of carbohydrates, organic acids, amino acids, halides, steroids, fatty acids, lipids, vitamins, nucleic acids and derivatives thereof, dopamine, L-dopa, serotonin, and epinephrine;
n is 1 to 30; and
F is a fluorochrome molecule having absorption and emission maximum between 600 nm and 1200 nm.

47. A method of in vivo optical imaging, the method comprising:
(a) administering to a subject an optical imaging probe of claim 46;
(b) allowing time for the optical imaging probe to reach the target tissue;
(c) illuminating the target tissue with light of a wavelength absorbable by the optical imaging probe; and
(d) detecting the optical signal emitted by the optical imaging probe.

48. An optical imaging probe represented by general formula (I):
M(ₙ)-F (I)
wherein:
M is glucose or deoxyglucose;
n is 1 to 30; and
F is a fluorochrome molecule having absorption and emission maximum between 600 nm and 1200 nm.

49. A method of in vivo optical imaging, the method comprising:
(a) administering to a subject an optical imaging probe of claim 48;
(b) allowing time for the optical imaging probe to reach the target tissue;
(c) illuminating the target tissue with light of a wavelength absorbable by the optical imaging probe; and
(d) detecting the optical signal emitted by the optical imaging probe.
